(19) United States
(12) Patent Application Publication
(10) Pub. No.: US 2025/0151978 A1
(43) Pub. Date: May 15, 2025

(54) COLLISION WARNING FOR MEDICAL DEVICE

(71) Applicant: Canon U.S.A., Inc., Melville, NY (US)

(72) Inventors: Takahisa Kato, Brookline, MA (US); HuaLei Shelley Zhang, Brookline, MA (US); Brian Ninni, Woburn, MA (US)

(21) Appl. No.: 18/837,447

(22) PCT Filed: Feb. 7, 2023
(86) PCT No.: PCT/US2023/062149

§ 371 Date: Aug. 9, 2024

Related U.S. Application Data
(60) Provisional application No. 63/309,410, filed on Feb. 11, 2022.

(51) Int. Cl. A61B 1/00; A61B 1/005; A61B 1/05; A61B 1/267; A61B 34/10; A61B 34/20; A61M 25/01

(52) U.S. Cl. CPC A61B 1/00055; A61B 1/00045; A61B 1/00097; A61B 1/005; A61B 1/05; A61B 1/2676; A61B 34/20; A61B 2034/107; A61M 2025/0166

(57) ABSTRACT

A medical apparatus (100) includes a medical device (108) with bending sections and a distal end, at least one imaging device (110) at the distal end, at least one sensor (112), and at least one processor (201) which performs receiving input data input data from user input, the imaging device (110), the sensor (112), or combinations thereof, determining a bending plane and a bending angle of the distal end of the medical device (108), predicting location movement and position of the bending sections based on input data, displaying an image view based on the input data and displaying the predicted location movement and position of the bending sections on the image view.

### CROSS-REFERENCE TO RELATED APPLICATIONS

This application claims the benefit of priority of provisional U.S. Patent Application No. 63/309,410 filed on Feb. 11, 2022, which is hereby incorporated by reference herein in its entirety.

### TECHNICAL FIELD

The present disclosure generally relates to medical devices and, more particularly, to medical devices, apparatuses, methods, and storage mediums to provide collision warning and better guide a user to maneuver a medical device.

### BACKGROUND ART

Minimally-invasive imaging and therapeutic devices find use in image guided therapy to look inside a body. A catheter, endoscope, ablation device, and other devices can carry out these types of medical procedures, where a flexible medical tool is inserted into a patient's body and an instrument is passed through the tool to examine or treat an area inside the body. A bronchoscope is an endoscopic instrument to view inside the airways of a patient. Catheters and other medical tools can be inserted through a tool channel in the bronchoscope to provide a pathway to a target area in the patient for diagnosis, treatment, or the like.

A medical device in the form of a robotic or snake catheter assembly typically has a rotational drive assembly to impart rotational movement to a guide wire of a catheter. The drive assembly is releasably connected to the catheter and a breakaway mechanism can be used so the drive assembly disconnects from the catheter in response to a breakaway force.

During medical procedures, there are multiple ways to provide visual presentation to guide medical device navigation, such as endoscopes, catheters, or other medical devices. An over-the-shoulder view configuration in virtual bronchoscopy can be used with updated lung airway models from tomosynthesis fluoroscopy. A wide-angle panoramic view with virtual bronchoscopy can be used to assist in situations where a medical device can face an airway wall. Other endoscopic tools for a minimally invasive procedure, such as a catheter, can be equipped with multiple cameras to provide full angle 360° integrated view for full presentations of the surroundings.

U.S. Publication No. 20220202502 describes visualization and orientation guidance during an endoscopy procedure that provides peripheral navigation used to guide a navigation catheter in the tracheobronchial tree once the scope is wedged and cannot be advanced. Default views include a dynamic 3D map, bronchoscope, local, and tip views. A virtual panoramic view provides wide-angle vision for users during navigation and help in the wall-facing situation.

Problems currently exist during medical procedures with visual presentations to guide medical device navigation. For example, when a user maneuvers a robotic catheter to approach a turning corner around bifurcations inside a lung airway, the catheter body is likely to buckle. This buckling happens especially when the distal bending section hits the airway wall. The distal bending section is maneuvered to align with the airway shape, while the user views the endoscope camera. However, because the endoscope camera is distally away from the distal bending section due to the rigid tip in between, the user needs to compensate bending angles to avoid collisions of the catheter body with the airway wall.

Prior art peripheral navigation views are known that present visual aid of current surroundings from angles beyond the endoscope camera view, however, it is still difficult for users to predict potential collisions of the catheter body against the airway wall if maintaining the bending command and/or traveling along the centerline on the endoscope camera view at the rigid tip only. See, for example, U.S. Pat. Nos. 5,036,852, 6,827,695, 10,420,626, 10,893,911, 11,202,652, 11,558,653, and U.S. Publication Nos. 20130238048, 20150133959, and 20170188946.

It would be beneficial to overcome these concerns and implement solutions to avoid collisions of a medical device during medical procedures.

### SUMMARY

The present disclosure advantageously provides collision warnings and better guides a user to maneuver a medical device.

According to some embodiments, a medical apparatus includes a medical device with at least one bendable section, a distal end, at least one imaging device at the distal end, at least one sensor, and at least one processor which performs receiving input data input data from user input, an imaging device, a sensor, or combinations thereof, determining a bending plane and a bending angle of the distal end of the medical device, predicting location movement and position of the at least one bendable section based on the input data, displaying an image view based on the input data and displaying the predicted location movement and position of the at least one bendable section on the image view.

According to some embodiments, a method for a medical apparatus with a medical device with at least one bendable section, a distal end, at least one imaging device, and at least one sensor, the method includes receiving input data from user input, an imaging device, a sensor, or combinations thereof, from an imaging device and sensor, determining a bending plane and a bending angle of the distal end of the medical device, predicting location movement and position of the at least one bendable section based on the input data, displaying an image view based on the input data, and displaying the predicted location movement and position of the at least one bendable section on the image view.

Further features of the present disclosure will become apparent from the following description of exemplary embodiments with reference to the attached drawings.

### BRIEF DESCRIPTION OF THE DRAWINGS

FIG. 1 illustrates a medical apparatus according to some embodiments.

FIG. 2 is a block diagram of the medical apparatus of FIG. 1.

FIG. 3 is a block diagram of a controller according to some embodiments.

FIGS. 4A and 4B illustrate a catheter according to some embodiments.

FIG. 5 illustrates a method according to some embodiments.

FIG. 6 illustrates a steerable catheter with an endoscopic camera at the distal end according to some embodiments.

FIG. 7, which includes FIGS. 7A, 7B, and 7C, illustrates a steerable catheter with an endoscopic camera at the distal end according to some embodiments.

FIG. 8 is a top view of the distal section of the steerable catheter according to some embodiments.

FIG. 9 is a top view of the distal section of the steerable catheter according to some embodiments.

FIG. 10 illustrates a Follow-the-Leader trajectory according to some embodiments.

### DESCRIPTION OF THE EMBODIMENTS

Various exemplary embodiments, features, and aspects of the disclosure that relate to medical devices, apparatuses, methods, and storage mediums to provide collision warning and better guide a user to maneuver a medical device will be described below with reference to the drawings that may have different characteristics, advantages, disadvantages, performance parameters, or the like.

In order to better guide a user to maneuver a medical device, such as a catheter around bifurcations in the airway, a future position of the distal bending tip (or one or more selected locations along the catheter body) can be predicted based on current bending angle and plane, by assuming certain insertion/travel distance and rigid tip length. The predicted position will be projected onto current endoscopic camera view or virtual bronchoscope view. In conjunction with delineation of lumen opening versus airway wall in the present view, the user will be informed whether current bending command via joystick input or other type of input is sufficient for the catheter body to traverse through the opening space or result in a collision against the airway wall. If the latter happens, the user will adjust the bending command, particularly compensating the bending angle, until the predicted position of bending section tip lands within the lumen opening space of the current endoscope camera view or virtual bronchoscope view.

In the following embodiments, medical apparatus, equipment, device or instrument configurations to avoid collisions of a medical device during medical procedures are described that functionally implement intravascular imaging modalities including, for example, CT (computed tomography), MRI (magnetic resonance imaging), IVUS (intravascular ultrasound), positron emission tomography (PET), X-ray imaging, angiography, optical coherence tomography (OCT), multi-modality OCT (MMOCT), near infrared auto fluorescence (NIRAF), spectrally encoded endoscopy (SEE), combinations or hybrids thereof, or the like. The medical apparatus configurations that are described may have different characteristics, advantages, disadvantages, performance parameters, or the like. The present disclosure is not limited to any particular configuration.

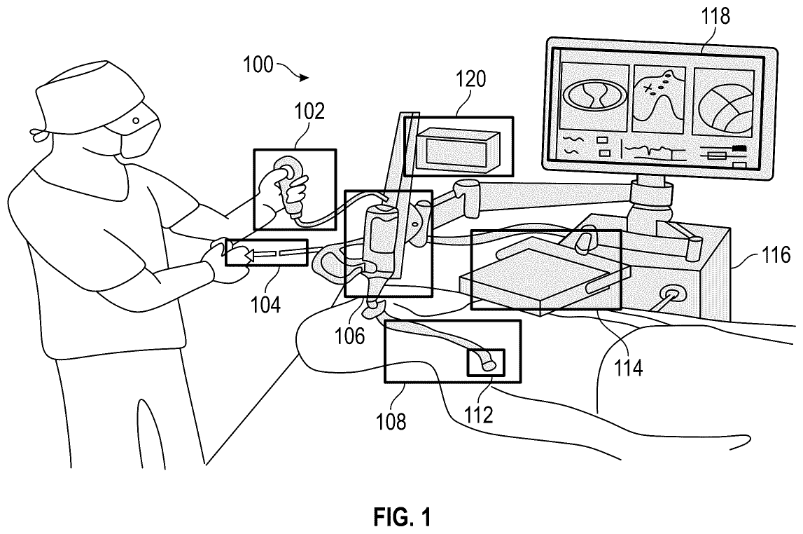

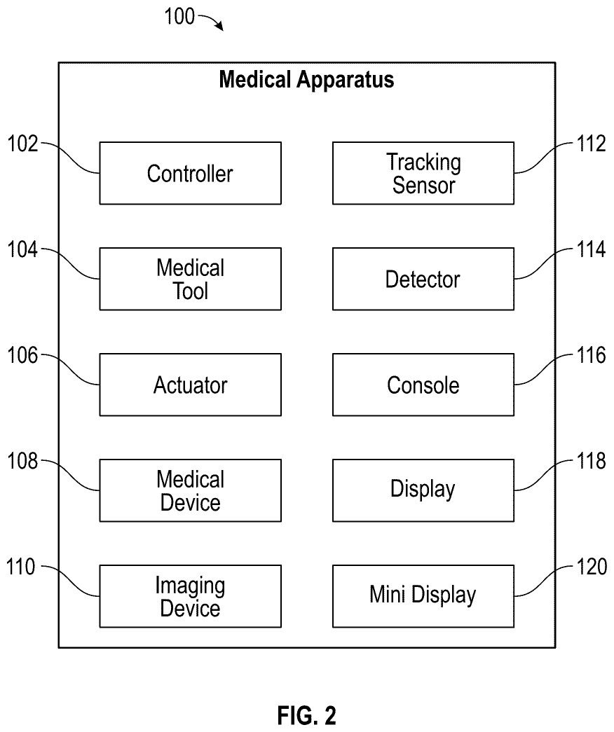

FIG. 1 illustrates a medical apparatus 100 configured as an exemplary robotic catheter assembly or snake configuration according to some embodiments. FIG. 2 shows a hardware configuration of the medical apparatus 100. The medical apparatus 100 is not limited to these arrangements and variations or other configurations of the medical apparatus 100 are within the scope of the present disclosure.

The robotic catheter 100 includes one or more of a hand-held controller 102, a medical tool 104, an actuator 106, a medical device 108, an imaging device 110, a sensor 112, a detector 114, a console 116, a display 118, and a mini display 120, and can include other elements or components. Throughout the present disclosure, the medical tool 104 is referred to as a “biopsy tool” and the medical device 108 is referred to as a “catheter”, but these are exemplary and one or more of a variety of other types of tools, devices, configurations, or arrangements also falls within the scope of the present disclosure including, for example, snake robotic catheter, an endoscope, a sheath, guidewire, needle, probe, forceps, or the like.

The robotic catheter 100 according to some embodiments can implement functioning through use of one or more processes, techniques, algorithms, or the like, that can to avoid collisions of a medical device while providing better work efficiency to physicians during a medical procedure.

The controller 102 has a housing with an elongated handle or handle section which can be manually grasped, and one or more input devices including, for example, a lever or a button or another input device that allows a user, such as a physician, to send a command to the medical apparatus 100 to move the catheter 108. The controller 102 executes software, computer instructions, algorithms, or the like, so a user can complete all operations with the hand-held controller 102 by holding it with one hand.

The medical tool 104 can be a biopsy tool or other type of tool. The actuator 106 can include one or more motors and drives each section of the catheter 108. The controller 102, medical device 108, console 116, and other elements are interconnected to the actuator 106. The controller 102 includes at least one processor and is configured to control the medical device 108 through the actuator 106, and to control the actuator 106 in accordance with the manipulation by a user.

The medical device 108 is configured as a catheter or another type of medical device. The imaging device 110 is a mechanical, digital, or electronic device configured to record, store, or transmit visual images, e.g. a camera, camcorder, motion picture camera, or the like. The sensor 112 can be an electromagnetic tracking sensor (EM tracking sensor) and is attached to the tip of the catheter 108. The detector 114 detects a position of the EM tracking sensor 112 and outputs the detected positional information to the controller 102 and/or the console 116. The controller 102 receives the positional information of the catheter tip directly from the tracking sensor 112 or from the detector 114.

The console 114 executes software, computer instructions, algorithms, or the like, and controls to display a navigation screen on the display 118 and other types of imagery or information on the mini-display 120. The console 116 can generate a three-dimensional (3D) model of an internal branching structure, for example, lungs or other internal structures, of a patient based on medical images such as CT, MRI, or the like. Alternatively, the 3D model may be received by the console 114 from another device.

The console 116 acquires catheter position information from the detector 114. Alternatively, the console 116 can acquire the catheter position information directly from the tracking sensor 112.

The console 116 generates and outputs the navigation screen to the display 104 based on the 3D model and the catheter positional information by executing the software. The navigation screen can indicate a current position of the catheter 108 on the 3D model. By the navigation screen, a user can recognize the current position of the catheter 110 in the branching structure.

The console 116 can execute a correction of the acquired 3D model based on the positional information of the catheter 108 so as to minimize a divergence between the position of the catheter 108 and a path mapped out on the 3D model.

The display 118 and/or the mini display 120 can be a display device configured, for example, as a monitor, an LCD (liquid-crystal display), an LED (light-emitting diode) display, an OLED (organic LED) display, a plasma display, an organic electro luminescence panel, or the like. Based on the control of the apparatus, the navigation screen may be displayed on the display 116 showing one or more images being captured, captured images, captured moving images recorded on the storage unit, or the like. The mini display 120 is smaller than the display 118 and can they can each display similar or other types of imagery and/or information.

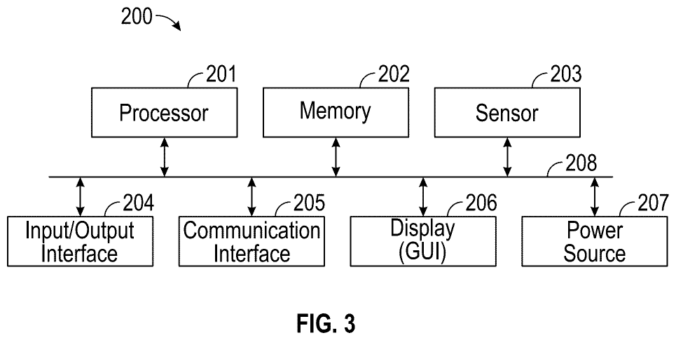

The controller 102 and/or the console 116 can include one or more or a combination of levers, keys, buttons, switches, a mouse, a keyboard, or the like, to control the elements of the apparatus 100 and each has configurational components 200, as shown in FIG. 3, that include one or more or a combination of a processor 201, a memory 202, a sensor 203, an input and output (I/O) interface 204, a communication interface 205, a display 206, a power source 207, and can include other elements or components. The apparatus 100 can be interconnected with medical instruments or a variety of other devices, and can be controlled independently, externally, or remotely by the controller 102 and/or the console 116.

The processor 201 is configured as one or more processors, control circuit, circuitry, or combinations thereof, and performs overall control of the medical apparatus 100. The memory 202 stores the program, software, computer instructions, information, other data, or combinations thereof. The memory 202 is used as a work memory. The processor 201 executes a program, instructions, code or software stored or developed in the memory 202 to perform various data processing, computation, algorithmic tasks, or other functions of the medical apparatus 100.

The sensor 203 monitors, measures or detects various types of data of the medical apparatus 100, and can transmit or send the sensor readings or data to a host through a network. The I/O interface 204 can interconnect various components with the medical apparatus 100 to transfer data or information to or from the medical apparatus 100. The I/O interface 204 can input the catheter positional information to the console 116 and can output information for displaying a navigation screen to the display 118. The communication interface 205 can interconnect various components with the medical apparatus 100 to facilitate communication to or from or the medical apparatus 100.

The display 206 corresponds to the display 118 and/or the mini display 120 and can present a display to a user to view images, data or other information, and can be configured as an LCD or other type of display. The controller 102 and/or the console 116 can perform display control of the display 206 and control of input of various kinds of setting or default information set by the input/output interface 204 and the communication interface 205, and to provide inputs to the medical apparatus 100.

The power source 207 provides power to the medical apparatus 100 to maintain a regulated power supply to the medical apparatus 100, and can operate in a power-on mode, a power-off mode, and can operate in other modes. The power source 107 can include a battery contained in the medical apparatus 100 and can include an external power source such as line power or AC power from a power outlet that can interconnect with the medical apparatus 100 through an AC/DC adapter and a DC/DC converter, or an AC/DC converter in order to adapt the power voltage from a source into one or more voltages used by components in the medical apparatus 100.

The components are connected together by a bus 208 so that the components can communicate with each other. The bus 208 connects the medical apparatus 100 to input devices, output devices, communication devices, or other devices. The input devices are configured to enable the user to communicate information and select commands to the medical apparatus 100, and can include one or more or a combination of a mouse, keyboard, touchscreen, or the like, with keys or buttons with alphanumeric, icon, emoji, or other types of symbols. The output devices are configured to display data or images generated by the medical apparatus 100, and can include printers, display devices, or other output configurations.

The bus 208 transmits and receives data between these pieces of hardware connected together, or transmits a command from the processor 201 to the other pieces of hardware. The components can be implemented by one or more physical devices that may be coupled to the processor 201 through a communication channel. For example, the controller 102 and/or the console 116 can be implemented using circuitry in the form of ASIC (application specific integrated circuits) or the like. Alternatively, the controller 102 and/or the console 116 can be implemented as a combination of hardware and software, where the software is loaded into a processor from a memory or over a network connection. Functionality of the controller 102 and/or the console 116 can be stored on a storage medium, which may include RAM (random-access memory), ROM (read only memory), magnetic or optical drive, diskette, cloud storage, or the like.

The sensor 203 includes one or more or a combination of a processor, detection circuitry, memory, hardware, software, firmware, and can include other circuitry, elements, or components. The sensor 203 can be a plurality of sensors and acquires sensor information output from one or more sensors that detect motion, current position and movement of components interconnected with the medical apparatus 100. The sensor 203 can include a multi-axis acceleration or accelerometer sensor and a multi-axis gyroscope sensor, can be a combination of an acceleration and gyroscope sensors, can include other sensors, and can be configured through the use of a piezoelectric transducer, a mechanical switch, a single axis accelerometer, a multi-axis accelerometer, or other types of configurations. The sensor 203 can monitor, detect, measure, record, or store physical, operational, quantifiable data or other characteristic parameters of the medical apparatus 100 including one or more or a combination of an impact, shock, drop, fall, movement, acceleration, deceleration, velocity, rotation, temperature, pressure position, orientation, motion, or other types of data of the medical apparatus 100 in multiple axes, in a multi-dimensional manner, along an x axis, y axis, z axis, or any combination thereof, and can generate sensor readings, information, data, a digital signal, an electronic signal, or other types of information corresponding to the detected state. The medical apparatus 100 can transmit or send the sensor reading data wirelessly or in a wired manner to a remote host or server. The sensor 203 can be interrogated and can generate a sensor reading signal or information that can be processed in real time, stored, post processed at a later time, or combinations thereof. The information or data that is generated by the sensor 203 can be processed, demodulated, filtered, or conditioned to remove noise or other types of signals. The sensor 203 includes one or more or a combination of an acceleration, deceleration, or accelerometer sensor, a gyroscope sensor, a power sensor, a battery sensor, a proximity sensor, a motion sensor, a position sensor, a rotation sensor, a magnetic sensor, a barometric sensor, an illumination sensor, a pressure sensor, an angular position sensor, a temperature sensor, an altimeter sensor, an infrared sensor, a sound sensor, an air monitoring sensor, a piezoelectric sensor, a strain gauge sensor, a sound sensor, a vibration sensor, a depth sensor, and can include other types of sensors.

The acceleration sensor, for example, can sense or measure the displacement of mass of a component of the medical apparatus 100 with a position or sense the speed of a motion of the component of the medical apparatus 100. The gyroscope sensor can sense or measure angular velocity or an angle of motion and can measure movement of the medical apparatus 100 in up to six total degrees of freedom in three-dimensional space including three degrees of translation freedom along cartesian x, y, and z coordinates and orientation changes between those axes through rotation along one or more or of a yaw axis, a pitch axis, a roll axis, and a horizontal axis. Yaw is when the component of the medical apparatus 100 twists left or right on a vertical axis. Rotation on the front-to-back axis is called roll. Rotation from side to side is called pitch. The sensor 203 can monitor shock or drop impact with low power consumption, dynamic range, and bandwidth to accurately detect and capture shock events and convert the sensor readings to a digital signal for additional or post processing. An entire shock profile can be characterized by its peak amplitude and pulse width for further analysis. The processor 201 of the medical apparatus 100 can also interrogate the capacity of the power source 207, and can warn a user to replace the battery at a time when a value of the battery capacity falls below a predetermined threshold amount.

The acceleration sensor can include, for example, a gravity sensor, a drop detection sensor, or the like. The gyroscope sensor can include an angular velocity sensor, a hand-shake correction sensor, a geomagnetism sensor, or the like. The position sensor can be a global positioning system (GPS) sensor that receives data output from a GPS. The longitudinal and latitude of a current position can be obtained from access points of a radio frequency identification device (RFID) and a WiFi device and information output from wireless base stations, for example, so that these detections may be used as position sensors. These sensors can be arranged internally or externally of the medical apparatus 100.

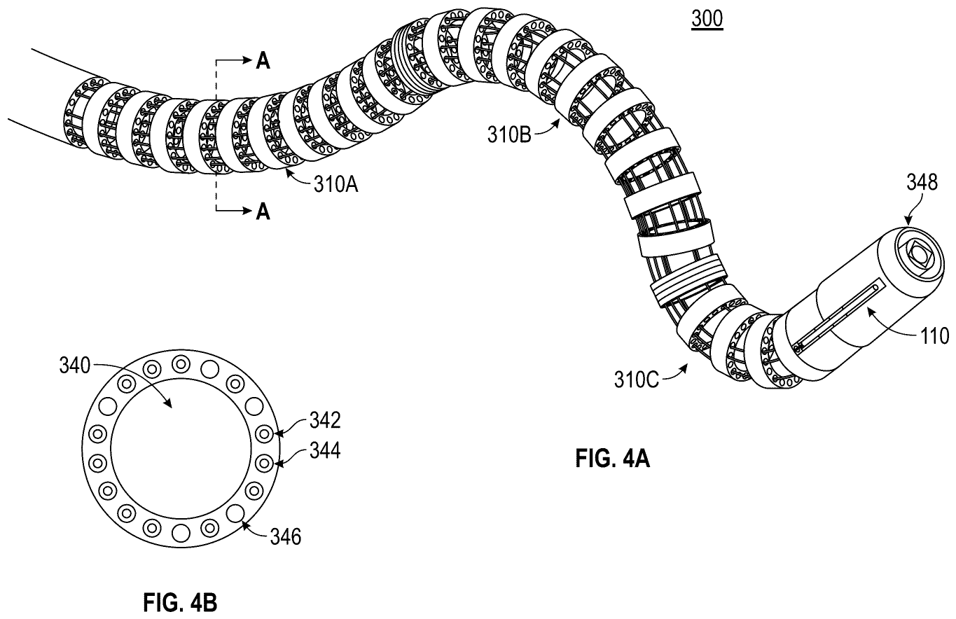

The medical device 108 according to some embodiments can be configured as a catheter 300, as shown in FIGS. 4A and 4B.

The catheter 300 is a flexible medical device with bendable sections including a proximal bendable section 310A, a middle bendable section 310B, and a distal bendable section 310C. Running proximal to distal through the catheter 300 is hollow chamber 340 that can be used as a working channel for medical procedures. The catheter 300 includes a plurality of driving wires 342 and supporting wires 344 that are each located in lumen 346 surrounding the central hollow chamber 340, as shown in the cross-sectional view of FIG. 4B. Each of the proximal bendable section 310A, middle bendable section 310B, and distal bendable section 310C of the catheter 300 can be bent by the plurality of driving wires 342 (driving linear members) as driving backbones. The posture of the catheter 300 can be maintained by the supporting wires 344 (supporting linear members) as passive sliding backbones. One or more lumen can be left free to facilitate the use of additional optical fibers or wires to be added to the catheter 300. The tracking sensor 110 is attached to the atraumatic tip 348 of the catheter 300. At the proximal end of the catheter 300, the driving wires are connected to the actuator 106. The actuator 106 can include one or more motors and drives each section of the catheter 300 by pushing and/or pulling the driving wires.

The controller 102 can control the catheter 108 based on an algorithm known as follow-the-leader (FTL) algorithm or other algorithms. By applying the FTL algorithm, the middle section 310B and the proximal section 310A of the catheter 108 move at a first position in the same way as the distal section moved at the first position or a second position near the first position. The controller 102 and/or the console 114 can include an input element to allow a user to positionally adjust the flexible portions of the catheter 108. The input element may be configured as a mouse, a keyboard, joystick, lever, or another shape to facilitate user interaction.

The medical apparatus 100 provides collision warning and better guides a user to maneuver the medical device 108, and is configured to predict movement of the medical device 108 based on user input, the imaging device 110, the sensor 112, or combinations thereof, as well as previous predictions made by the medical apparatus 100. The user can adjust the pose of sections other than the tip, and can also predict the tip dislocation, or combination of stage motion and section articulation.

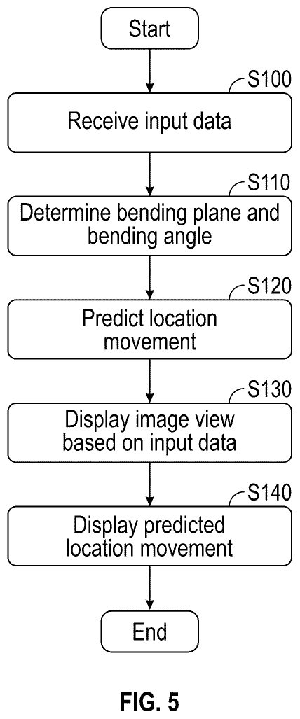

FIG. 5 shows a method according to some embodiments for the medical apparatus 100 with the medical device 108 with bending sections 310 and a distal end 348, at least one imaging device 110, and at least one sensor 112. In step S100, the method receives input data from user input, the imaging device 110, the sensor 112, or combinations thereof. In step S110, the method determines a bending plane and a bending angle of the distal end of the medical device. In step S120, the method predicts location movement and position of the bending sections based on the input data. In step S130, the method displays an image view based on the input data, and displays the predicted location movement and position of the bending sections on the image view.

The method can also detect collision of the medical device based on the input data, provide a collision warning of the medical device based on the input data, or limit a bending angle of the distal end of the medical device to avoid future collisions with an airway wall.

The method can display a constant arc by assuming an insertion distance so that a tip of the distal bending section can be projected onto the bending plane wherein the predicted location movement and position of the bendable sections is based on an insertion distance and a length of the rigid tip. The constant arc can be represented by an intersection line on the image view.

The method provides collision warning and better guides a user to maneuver a medical device, and is configured to predict medical device movement based on user input, the imaging device 110, the sensor 112, or combinations thereof, as well as previous predictions made by the medical apparatus 100. The user can adjust the pose of sections other than the tip, and can also predict the tip dislocation, or combination of stage motion and section articulation. In other words, the medical apparatus 100 can simulate any sort of user adjustment from the current point to visualize how it will affect the tip of the medical device.

The user could then, after finding a satisfactory input or a collection of inputs, simply automatically send them to the controller rather than have to repeat them manually. This can be done incrementally or continuously.

In a case where the bendable medical device or catheter has different length bending sections, the position of the end effector of a following section may not match the position of the tip end effector at the same location. This can be problematic and the medical apparatus 100 can take this into consideration when calculating the predicted motion.

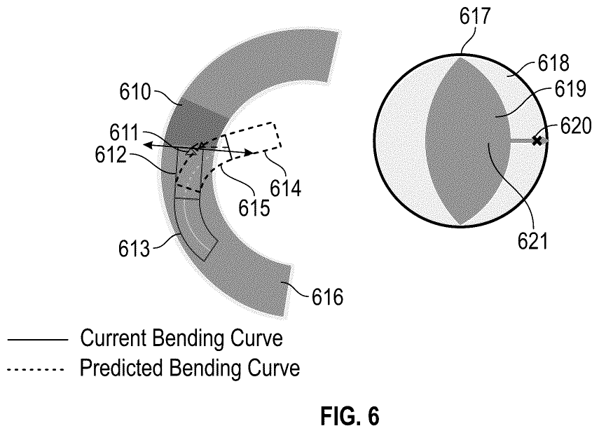

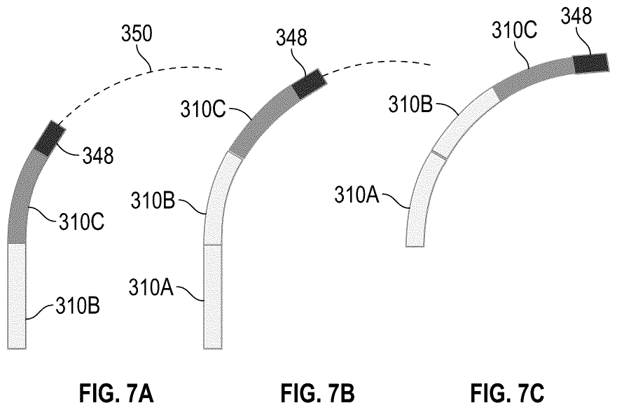

The medical device and the catheter of FIGS. 1-4B can avoid collisions of the medical device during medical procedures that are described below with reference to FIGS. 6 and 7A-7C that illustrate a steerable catheter with an endoscopic camera at the distal end according to some embodiments.

FIG. 6 shows an endoscope illumination and image capture range 610, and endoscope lens 611, a rigid tip (current) 612, a distal bendable section (current) 613, a rigid tip (predicted) 614, a distal bendable section (predicted) 615, an airway 616, an endoscope camera view 617, an airway wall 618, an open space 619, a predicted distal bendable section tip 620, and a bending plane (intersected as line) 621.

In order to better guide a user to maneuver the catheter 300 around bifurcations in an airway, a future position of the distal bendable section tip (or one or more selected locations along a catheter body) can be predicted based on a current bending angle and plane, by assuming certain insertion/travel distance and rigid tip length. The predicted position can be projected onto a current endoscope camera view or virtual bronchoscope view. In conjunction with delineation of a lumen opening versus an airway wall in the present view, the user can be informed whether a current bending command via input, such as a joystick input, is sufficient for the catheter body to traverse through the opening space or result in a collision against airway wall. If the latter happens, the user can adjust the bending command, particularly compensating the bending angle, until the predicted position of bendable section tip lands within the lumen opening space of the current endoscope camera view or virtual bronchoscope view.

The steerable catheter 300 of FIGS. 4A and 4B includes proximal, middle, and distal bendable sections 310A, 310B, 310C, and a rigid tip with an endoscopic camera at the distal end 348 (FIGS. 6 and 7). The operator can steer the distal bendable section 310C three-dimensionally and insert/remove the steerable catheter 300 by commanding the robotic sub-system with a joystick. When the bendable sections bend, the rigid tip at the distal end 348 of the distal bendable section 310C does not bend and keeps its straight shape (FIG. 6). The robotic catheter 300 can display a real-time endoscopic view with the endoscopic camera and the robot status information, i.e., the current bending angle and bending plane, the current insertion amount etc. Specifically, the operator can check the endoscopic view and the robot status information and decide the bending angle, orientation and insertion and removal of the catheter 300 to navigate through confined space in the anatomy, i.e., lung airways.

A robot feature on Follow-the-Leader motion allows achieving a snake-like insertion following the curved pathway, because proximal and middle bendable sections 310A, 310B will follow the distal bendable section 310C with continuing arc trajectory (FIG. 7). In the follow-the-leader motion, the operator can bend the distal bendable section 310C with the rigid tip 348 by using the joystick (FIG. 7A). At that time, the operator can check the real-time endoscopic view and control the bending orientation and amount. For example, in FIG. 7, when the operator would like to follow the curved pathway with a dotted line in FIG. 6, the operator would like to match the bending angle of the distal bendable section 310C (FIG. 7A), then insert the steerable catheter 300. When the middle bendable section 310B reaches the previous position of the distal bendable section 310C, the controller automatically bends the middle section 310B to the same amount as the distal bendable section 310C in the previous position (FIG. 7B). During this insertion, the operator can also continue to adjust bending of the distal bendable section 310C to the dotted line. By continuing this insertion, the distal bendable section 310C can be also automatically bent with the same angle of the middle bendable section 310B in the previous position (FIG. 7B). With these steps, the steerable catheter 300 can follow the curved pathways by minimizing the risk to hit the wall of the anatomy. In these steps, the operator can control only the distal bendable section 310C while the controller covers the control of the other bendable sections 310A, 310B automatically.

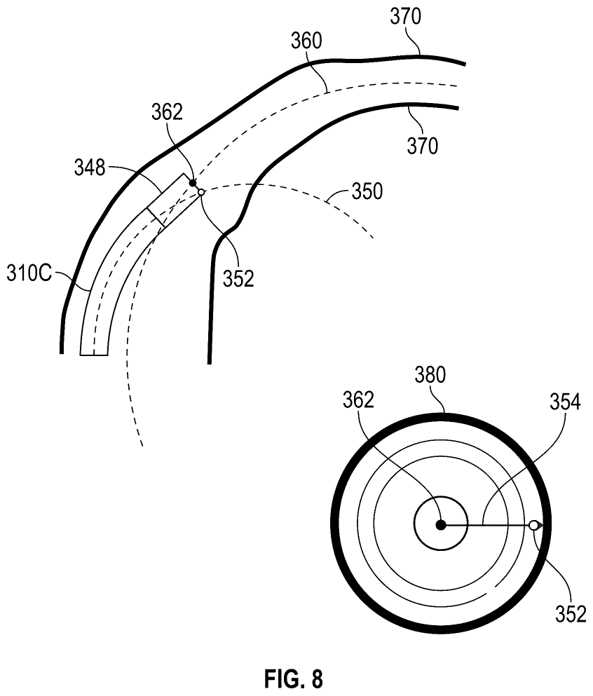

FIG. 8 is a top view of the distal bendable section 310C with the rigid tip 348 and the anatomy to show the relationship between the center line of the airways 360 and the trajectory 350 the steerable catheter will follow with the follow-the-leader motion. Specifically, FIG. 8 also shows the endoscope view center 362 at the distal end of the rigid tip 310C as well as the real-time display 380 of the endoscopic view 380 on the display at the bottom of FIG. 8.

In FIG. 8, the operator can adjust the endoscopic view center 362 to the center line 360 of the airways by adjusting the center of the real-time endoscopic view 380 to the center of the anatomic feature of the airway 360 on the display. In this situation, since the endoscopic view locates at the distal end of the rigid tip 348 instead of the distal end of the distal bendable section 310C, the bending angle of the distal bendable section 310C results in mismatching to the center line of the anatomy. If the operator continues the insertion, the steerable catheter 300 will follow the dotted line 350 with the follow-the-leader motion and would collide with the airway wall 370 even when the operator tries to adjust the view center 362 of the endoscope with the anatomy center. This makes the follow-the-leader control counterintuitive for the operator.

According to some embodiments, the controller 200 can compute the dotted line 350 by using the robot kinematic and the distal bending angle/orientation as a prediction of the follow-the-leader trajectory and determine the position of the distal end of the distal section (the circle in FIG. 8) as a prediction of the bending-section tip location 352 along the dotted line 350. To determine the circle 352, the operator can select the perpendicular distance from the view center 362, then the controller 200 actually determines the one white dot 352 and displays the white dot 352 on the real-time endoscopic view 380. With the display of the white dot 352 on the real-time endoscope view 380, the controller 200 can apply the conversion ratio of the distance between the distance on the endoscopic view and the computed Euclidean distance. The operator can use this white dot 352 as a reference to determine the bending angle 354 to match the center line 360 of the airways.

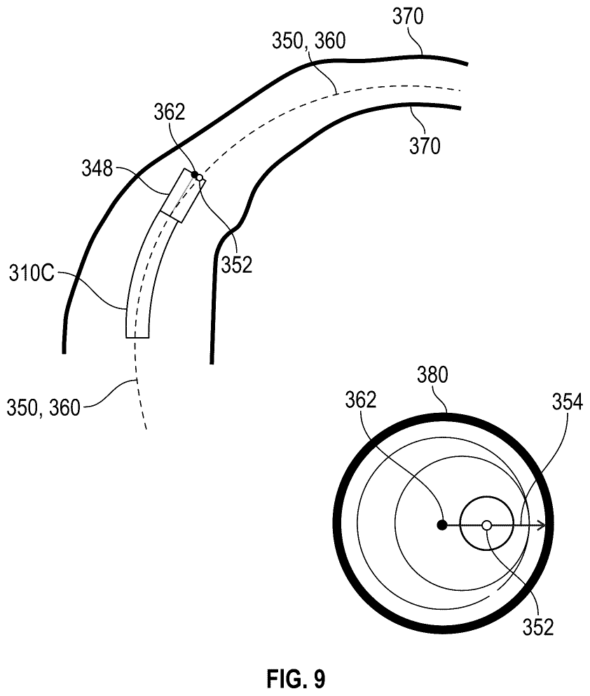

FIG. 9 is a similar top view of the distal section with the rigid tip 310C and the anatomy, but describes the situation when the prediction of the follow-the-leader trajectory 350 matches the center line 360 of the airways. In this situation, the view center 362 has offset from the airway in the real-time endoscope view 380 (FIG. 9 bottom) but the prediction of the bending-section tip location (white circle 352) is on the airways' center. Like this way, the operator can use the white dot 352 in the real-time endoscope view 380 to adjust the bending section angle 354 to match the center line 360 of the airway.

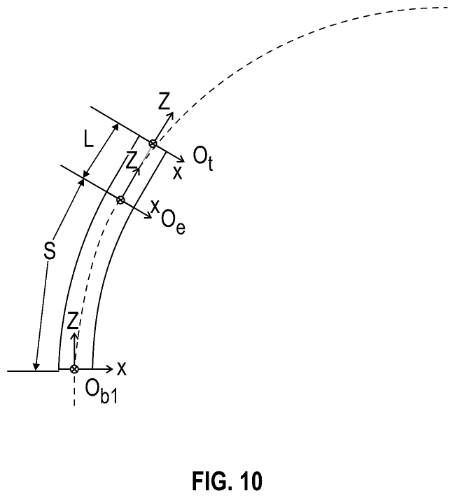

To compute the prediction of the follow-the-leader trajectory and the prediction of the bending-section tip location, the controller uses the three coordinate systems (FIG. 10). The endoscope view is on the endoscope coordinate (Oₜ). To display the view center (the black circle) on the real-time image, the controller can expect the black circle on the origin of coordinate Oₜ. The bending-section tip coordinate (Oₑ) are on the tip of the distal bending section. Since the rigid tip maintains straight shape always, the transformation between coordinates Oₜ and Oₑ can be computed.

The bending plane orientation and the bending angle can be measured by in the following manner. This information is associated to the distal section coordinate (O_{b1}). To compute the prediction of the follow-the-leader trajectory, the following two items are assumed.

The bending section shape has a constant curvature.

The prediction of the follow-the-leader trajectory is extrapolation of the constant curve shape of the bending section.

With the first assumption, the transformation between the coordinates of Oₑ and O_{b1} can be computed with the measured bending plane and bending angle with a known section length. Also, this assumption confirms the Z axis of the coordinate Oₑ is a tangent of the extrapolation of the constant curve shape of the bending section. Therefore, the controller 200 can compute the prediction of the follow-the-leader trajectory based on the coordinate of Oₜ and determine the prediction of the bending-section tip location along the determined trajectory.

As described above, the medical apparatus 100 provides solutions to problems that commonly exist during medical procedures with visual presentations to guide medical device navigation. The medical apparatus 100 provide collision warnings and better guides a user to maneuver a medical device.

The medical apparatus 100 includes a medical device, such as the catheter 300, with bendable sections 310A, 310B, 310C and a distal end, at least one imaging device at the distal end, at least one sensor, and a controller 200 which can receive input data from an imaging device and sensor. The controller 200 can determine a bending plane and a bending angle of the distal end of the catheter 300, predict location movement and position of the bendable sections based on the input data, display an image view based on the input data, and display the predicted location movement and position of the bendable sections on the image view.

According to some embodiments, the medical apparatus 100 provides collision warning and better guides a user to maneuver a medical device, and is configured to predict medical device movement based on input data from user input, the imaging device 110, a sensor 112, or combinations thereof, as well as previous predictions made by the medical apparatus 100. In other words, the medical apparatus 100 can simulate any sort of user adjustment from the current point to visualize how it will affect the tip of the medical device.

The user could then, after finding a satisfactory input or a collection of inputs, simply automatically send them to the controller rather than have to repeat them manually. This can be done incrementally or continuously.

In a case where the bendable medical device or catheter has different length bending sections, the position of the end effector of a following section may not match the position of the tip end effector at the same location. This can be problematic and the medical apparatus 100 can take this into consideration when calculating the predicted motion.

According to some embodiments, a medical apparatus is provided that includes a medical device with bendable sections and a distal end, at least one imaging device at the distal end, at least one sensor, and at least one processor which performs receiving input data from an imaging device and sensor, determining a bending plane and a bending angle of the distal end of the medical device, predicting location movement and position of the bendable sections based on the input data, displaying an image view based on the input data and displaying the predicted location movement and position of the bendable sections on the image view.

According to some embodiments, a method is provided for a medical apparatus with a medical device with bendable sections and a distal end, at least one imaging device, and at least one sensor, the method includes receiving input data from an imaging device and sensor, determining a bending plane and a bending angle of the distal end of the medical device, predicting location movement and position of the bendable sections based on the input data, displaying an image view based on the input data, and displaying the predicted location movement and position of the bendable sections on the image view.

The method can also detect collision of the medical device based on the input data, provide a collision warning of the medical device based on the input data, or limit a bending angle of the distal end of the medical device to avoid future collisions with an airway wall.

The medical device can be a steerable catheter, and a rigid tip can be at the distal end of the catheter. The rigid tip can be aligned with a normal vector of the distal bendable section. The predicted location movement and position of the bendable sections can be based on an insertion distance and a length of the rigid tip.

The method can display a constant arc by assuming an insertion distance so that a tip of the distal bendable section can be projected onto the bending plane wherein the predicted location movement and position of the bendable sections is based on an insertion distance and a length of the rigid tip. The constant arc can be represented by an intersection line on the image view.

The projection of the bendable section location onto the current virtual bronchoscopy viewpoint can be, for example, a birds-eye-view, an over-the-shoulder view, or other types of views, and can take into account the rigid tip length. Taking input of bending angle and bending plane, the current distal bendable section tip can be projected backward based on the rigid tip length, and then assuming the continuation of current bending angle, a constant arc can be projected forward by assuming an insertion distance, so that the bendable section tip can be projected onto the bending plane, represented by an intersection line on the endoscope camera view. A rigid tip axis can be aligned with normal vector of distal bending section, and the bending angles can be limited to avoid future collision with airway wall.

According to some embodiments, a storage medium is provided storing a program may be configured to cause a computer to execute to execute the method for a medical apparatus with a medical device with bendable sections and a distal end, at least one imaging device, and at least one sensor.

Additional features or aspects of present disclosure can also advantageously implement one or more AI (artificial intelligence) or machine learning algorithms, processes, techniques, or the like, to avoid collisions of a medical device during medical procedures. Such AI techniques use a neural network, a random forest algorithm, a cognitive computing system, a rules-based engine, or the like, and are trained based on a set of data to assess types of data and generate output. For example, a training algorithm can be configured to avoid collisions of a medical device during medical procedures. The model(s) can be configured as software that takes images as input and returns predictions for the given images as output. The model(s) can be an instance of a model architecture (set of parameter values) that has been obtained by model training and selection using a machine learning and/or optimization algorithm/process. A model can generally include, for example, an architecture defined by a source code (e.g. a convolutional neural network including layers of parameterized convolutional kernels and activation functions, or the like) and configuration values (parameters, weights, features, or the like) that are initially set to random values and are then over the course of the training iteratively optimized given data example, an objective function (loss function), an optimization algorithm (optimizer), or the like.

At least some of the medical images of detailed positional configurations of the patient anatomy relative to the catheter position can be used as input data and provided to the training algorithm. Initial images, output values and detailed positional configurations of the catheter position relative to the patient anatomy can be stored in a database to facilitate precise real-time correction of regional tissue deformation during an endoscopy procedure for new data. Through visualization guidance of device-to-image registration that are generated using input mapping to the model(s) or through expert research, machine learning can find parameters for AI processes. The training algorithm is configured to learn physical relationships in the input data to best describe these relationships or correlations. The data sets include information based on a number of factors including, for example, the acquired images, the number of acquired images, the angle of the image, the position of the image, detailed positional configurations of the medical device relative to the branching model, or the like. The data is evaluated using a weighted evaluation where the weights are learned through a training process, through subject matter specifications, or the like. Deep learning mechanisms can augment an AI process to identify indicators in the image data that can include, for example, new data images, output values or positional configurations of the catheter position relative to the patient anatomy, or the like.

Features of the present disclosure avoid collisions of a medical device during medical procedures.

### Other Embodiments

Embodiment(s) of the present disclosure can also be realized by a computerized configuration(s) of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computerized configuration(s) of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computerized configuration(s) may comprise one or more processors, one or more memories, circuitry, or a combination thereof (e.g., central processing unit (CPU), micro processing unit (MPU), or the like), and may include a network of separate computers or separate processors to read out and execute the computer executable instructions. The computer executable instructions may be provided to the computerized configuration(s), for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

1. A medical apparatus (100) comprising:
a medical device (108) with at least one bendable section (310) and a distal end (348);
at least one imaging device (110) at the distal end (348);
at least one sensor (203); and
a controller (200) which performs:
receiving input data from user input, the imaging device (110), the sensor (203), or combinations thereof;
determining a bending plane and a bending angle of the distal end (348) of the medical device (108);
predicting location movement and position of the at least one bendable section (310) based on the input data;
displaying an image view based on the input data; and
displaying the predicted location movement and position of the at least one bendable section (310) on the image view.

2. The medical apparatus (100) according to claim 1, wherein the controller (200) further performs detecting collision of the medical device (108) based on the input data.

3. The medical apparatus (100) according to claim 1, wherein the controller (200) further performs providing a collision warning of the medical device (108) based on the input data.

4. The medical apparatus (100) according to claim 1, wherein the controller (200) further performs limiting a bending angle of the distal end of the medical device to avoid future collisions with an airway wall.

5. The medical apparatus (100) according to claim 1, wherein the medical device (108) is a steerable catheter.

6. The medical apparatus (100) according to claim 1, wherein a rigid tip is at the distal end of the medical device (108).

7. The medical apparatus (100) according to claim 6, wherein the rigid tip is aligned with a normal vector of the at least one bendable section (310).

8. The medical apparatus (100) according to claim 6, wherein the predicted location movement and position of the at least one bendable section (310) is based on an insertion distance and a length of the rigid tip.

9. The medical apparatus (100) according to claim 6, further comprising displaying a constant arc by assuming an insertion distance so that a tip of the at least one bendable section (310) can be projected onto the bending plane wherein the predicted location movement and position of the at least one bendable section (310) is based on an insertion distance and a length of the rigid tip.

10. The medical apparatus (100) according to claim 9, wherein the constant arc is represented by an intersection line on the image view.

11. A method for a medical apparatus (100) with a medical device (108) with at least one bendable section (310) and a distal end (348), at least one imaging device (110), and at least one sensor (112), the method comprising:
receiving input data from user input, the imaging device (110), the sensor (112), or combinations thereof;
determining a bending plane and a bending angle of the distal end of the medical device (108);
predicting location movement and position of the at least one bendable section (310) based on the input data;
displaying an image view based on the input data; and
displaying the predicted location movement and position of the at least one bendable section (310) on the image view.

12. The method according to claim 11, further comprising detecting collision of the medical device (108) based on the input data.

13. The method according to claim 11, further comprising providing a collision warning of the medical device (108) based on the input data.

14. The method according to claim 11, further comprising limiting a bending angle of the distal end (348) of the medical device (108) to avoid future collisions with an airway wall.

15. The method according to claim 11, wherein the medical device (108) is a steerable catheter.

16. The method according to claim 11, wherein a rigid tip is at the distal end (348) of the medical device.

17. The method according to claim 16, wherein the rigid tip is aligned with a normal vector of the at least one distal bendable section (310).

18. The method according to claim 11, wherein the predicted location movement and position of the at least one bendable section (310) is based on an insertion distance and a length of the rigid tip.

19. The method according to claim 11, further comprising displaying a constant arc by assuming an insertion distance so that a tip (348) of the at least one bendable section (310) can be projected onto the bending plane wherein the predicted location movement and position of the at least one bendable section is based on an insertion distance and a length of the rigid tip.

20. The method according to claim 19, wherein the constant arc is represented by an intersection line on the image view.

21. A storage medium storing a program for causing a computer to execute a method for a medical apparatus (100) with a medical device (108) with at least one bendable section (310) and a distal end (348), at least one imaging device (110), and at least one sensor (112), the method comprising:
receiving input data input data from user input, the imaging device (110), the sensor (112), or combinations thereof;
determining a bending plane and a bending angle of the distal end (348) of the medical device (108);
predicting location movement and position of the at least one bendable section (310) based on the input data;
displaying an image view based on the input data; and
displaying the predicted location movement and position of the at least one bendable section (310) on the image view.
